(19) United States
(12) Patent Application Publication
(10) Pub. No.: US 2007/0055136 A1
(43) Pub. Date: Mar. 8, 2007

(54) Inspection apparatus using magnetic resonance

(75) Inventors: Yukari Yamamoto, Kunitachi (JP); Atsushi Maki, Fuchu (JP)

Correspondence Address: REED SMITH LLP, Suite 1400, 3110 Fairview Park Drive, Falls Church, VA, 22042, US

(73) Assignee: Hitachi, Ltd.

(21) Appl. No.: 11/471,708

(22) Filed: Jun. 21, 2006

(30) Foreign Application Priority Data
Aug. 24, 2005 (JP) 2005-241971

(51) Int. Cl. A61B 5/05

(52) U.S. Cl. 600/410

(57) ABSTRACT

A means to predict bone density reduction before it actually begins will be provided. This invention provides a method to detect abnormalities in the bone marrow hemopoietic cells which precede the bone density reduction, by obtaining a nuclear magnetic resonance image containing a flat bone or an a long bone epiphysis, and evaluating the signal intensity in the red marrow area.

### CLAIM OF PRIORITY

The present application claims priority from Japanese application JP 2005-241971 filed on Aug. 24, 2005, the content of which is hereby incorporated by reference into this application.

### FIELD OF THE INVENTION

This invention relates to an inspection apparatus using magnetic resonance to support prediction of bone density reduction and more particularly to an inspection apparatus using magnetic resonance to support prediction of bone density reduction by detecting abnormalities in hemopoietic tissues by a nuclear magnetic resonance image of bone marrow which contains hemopoietic cells.

### BACKGROUND OF THE INVENTION

Osteoporosis is a disease in which bone density (density of mineral contained in bone) decreases, and the bone gradually becomes fragile. In the bone, remodeling process exists wherein old bone tissues are absorbed and replaced by new bone tissues. When this process goes on appropriately, the bone remains to be healthy and can accommodate growth of the body during growth period.

When one is young, rate of ossification exceeds rate of bone resorption so that the bone density increases. With aging, rate of bone resorption begins to exceed that of ossification. When ossification is not sufficient, the bone density continues decreasing and results in osteoporosis.

It is being revealed that sex hormones are deeply involved in the increase and maintenance of the bone density. It is well known that a postmenopausal woman often develops osteoporosis. This is caused by reduced secretion of a female hormone, estrogen. The bone density may decrease not only in menopause but also other conditions in which the secretion of estrogen decreases (for example, excessively little food intake). It is being revealed that estrogen plays an important role in bone metabolism regardless of gender, from the findings obtained from male cases with an abnormality in estrogen receptors or male cases with an aromatase (steroid hormone) coloboma.

In diagnosis of osteoporosis, firstly, the presence of a fracture is examined by radiography, and osteoporosis is suspected when a fracture is detected in absence of heavy load. When no fracture is detected, the bone density is examined. X-ray or ultra sonography may be used for this examination, but the most typical method is dual energy X-ray absorptionmetry (the DXA method). In this method, the bone density is measured by irradiating two types of X-rays to the sites which are susceptible to a major fracture, such as a lumber vertebrae, a spine, and an articulation coxae. By using two types of X-rays, the effect of soft tissues can be eliminated and highly precise measurement can be achieved. The details of this technique are described in non-patent document 1: D. Schellinger, et al., “American Journal of Roentgenology 183, 1761-1765 (2004)”. Quantitative ultrasound (the QUS method) which uses ultra sonography is also used widely (non-patent document 2: J. Wehbe, et al., “Journal of Musculoskeletal & Neuron Interactions 3(3), 232-239 (2003)”). In this method, the bone density cannot be measured. Instead, by irradiating ultrasonic on calcaneum, ossein is calculated from acoustic velocity and attenuation in strength of the ultrasonic. However, the kind of physical quantity that ossein represents is not clear. Since the QUS method does not use X-ray, the method can be applied to a pregnant woman. However, since measurement precision of the QUS method is not sufficient, the DXA method is used as a standard measurement method.

As the DXA method widely used has a problem of X-ray contamination, researches are underway on bone density measurements using MRI (magnetic resonance imaging) which has a possibility to provide safe and repeated measurement. Non-patent document 3: F. W. Wehrli, et al., “Radiology 196, 631-641 (1995)” describes a method to diagnose osteoporosis from change in transverse relaxation speed R2*of nuclear magnetization, based on the difference in permeability between the cancellous bone and the bone marrow. In a patient with osteoporosis, it is indicated that the R2*of the bone marrow of the spine is lower than that of a healthy control. Non-patent document 4: S. Majumdar, et al., “European Radiology 7, S51-S55 (1997)” describes a method to directly observe structure of the cancellous bone and the bone marrow with use of micro imaging having high spatial resolution. It is indicated that the structure of the cancellous bone disappears in a postmenopausal woman. Non-patent document 5: M. A. Fernandez-Seara, et al., “Magnetic Resonance in Medicine 46, 103-113 (2001)” describes a method to measure bone volume using a proton density image. It is indicated that the signal in the bone marrow increases of the calcaneum if there is a decrease in the bone density. This increase in a signal may be attributable to increase in the amount of moisture in the calcaneum caused by the bone density reduction. A patent relating to this method has been applied (Patent document 1: JP-A No. 52008/2002). This patent relates to a MRI exclusively used for heel to determine the bone density, and employs the measurement method disclosed in the above non-patent document 5.

There is no symptom in early stage of osteoporosis, and even in later stage, no subjective symptom may appear at all. When deformation or fracture of a bone occurs, symptoms, such as a pain or deformation of the body, will develop. Since the bone has become fragile, the patient may easily suffer a fracture by a light load or a fall, and recovery from the fracture takes longer time. Depending on the site of the fracture, the patient cannot lead an independent life.

For treatment of osteoporosis, the patient is recommended to take more calcium and vitamin D, and receives a drug which has an effect to increase the bone density or to suppress the bone resorption. However, it is not easy to recover the bone density once it has lost. Therefore, it is important to maintain and increase the bone density by taking appropriate nutrition and exercise before the bone density really begins to decrease.

### SUMMARY OF THE INVENTION

The dual energy X-ray absorptionmetry as a standard method to measure bone density suffers from a disadvantage that it cannot provide safe and repeated examination because it uses X-ray irradiation. The QUS method using ultra sonography and the methods using MRI (nonpatent documents 3 to 5) are safer than the DXA method. However, both of these methods detect the change in signals caused by bone density reduction. This is same for the DXA method.

However, as mentioned above, it is not easy to recover the bone density once it has decreased. The patient must actively take calcium and vitamin D, and a drug which increases the bone density or suppresses the bone resorption for a long period of time. And as the patient easily suffers a fracture, he/she must take care to avoid a fracture in large bones especially a lumber vertebra, a spine, or a femur.

In the light of this situation, the object of the invention is to provide a means to predict bone density reduction before it actually occurs, by detecting in vivo phenomena which precede the bone density reduction.

In order to attain the purpose, the invention detects abnormality in the bone marrow hemopoietic cells which precedes the bone density reduction, by acquiring a nuclear magnetic resonance image of an area containing a flat bone or an epiphysis of a long bone, and evaluating the signal intensity in the bone marrow area.

In this invention, different constructions are contemplated which uses different evaluation methods to evaluate the signal intensity in the bone marrow area. Examples are as follows.

(1) According to one aspect of the present invention, an inspection apparatus using magnetic resonance includes: a static magnetic field generating device which generates static magnetic field in an area in which an object is placed, a gradient magnetic field generating device which gives gradient to the static magnetic field generated by the static magnetic field generating device, a transmitter which applies RF pulses to the object, a receiver which receives the nuclear magnetic resonance signals generated from the object, a control device which controls operation of the static magnetic field generating device, the gradient magnetic field generating device, the transmitter, and the receiver, an processing device which compares 1) the intensity of the nuclear magnetic resonance signal in the bone marrow area of a flat bone or a long bone epiphysis, with 2) the intensity of the nuclear magnetic resonance signal in an area other than the bone marrow area, and a display device which displays the result of the calculation of the processing device.

(2) According to another aspect of the invention, an inspection apparatus using magnetic resonance includes: a static magnetic field generating device which generates static magnetic field in an area in which an object is placed, a gradient magnetic field generating device which gives gradient to the static magnetic field generated by the static magnetic field generating device, a transmitter which applies RF pulses to the object, a receiver which receives the nuclear magnetic resonance signal generated from the object, a control device which controls operation of the static magnetic field generating device, the gradient magnetic field generating device, the transmitter, and the receiver, an area extraction means which automatically extracts the bone marrow area of a flat bone or a long bone epiphysis from the nuclear magnetic resonance signals received by the receiver, and an processing device which compares the intensity of the nuclear magnetic resonance signals in the bone marrow area of the flat bone or the long bone epiphysis received by the receiver, with the mean value of the intensity of the nuclear magnetic resonance signals in the bone marrow area of the flat bone or the long bone epiphysis of a healthy control.

(3) According to yet another aspect of the invention, an inspection apparatus using magnetic resonance includes: a static magnetic field generating device which generates static magnetic field in an area in which an object is placed, a gradient magnetic field generating device which gives gradient to the static magnetic field generated by the static magnetic field generating device, a transmitter which applies RF pulse to the object, a receiver which receives the nuclear magnetic resonance signal generated from the object, a control device which controls operation of the static magnetic field generating device, the gradient magnetic field generating device, the transmitter, and the receiver, an area extraction means which automatically extracts the bone marrow area of the flat bone or the long bone epiphysis from the nuclear magnetic resonance signals received by the receiver, a storage means which stores the nuclear magnetic resonance signal received by the receiver, an processing device which compares the intensity of the nuclear magnetic resonance signal in the bone marrow area of the flat bone or the long bone epiphysis at a first measurement, with the intensity of the nuclear magnetic resonance signal in the bone marrow area of the flat bone or the long bone epiphysis at a second measurement.

By using the apparatus according to the invention, it is possible to detect abnormality in the bone marrow hemopoietic cells which precedes the bone density reduction, and to predict the bone density reduction before it actually occurs.

### BRIEF DESCRIPTION OF THE DRAWINGS

FIG. 1 is an example of a flow chart illustrating an inspection method according to an embodiment of this invention;

FIG. 2 shows proton density images of males' heads;

FIG. 3 shows proton density images of females' heads;

FIG. 4 is a block diagram illustrating the inspection apparatus for carrying out the invention;

FIG. 5 is another example of a flow chart illustrating an inspection method according to an embodiment of this invention;

FIG. 6 is an example of a screen structure of an inspection apparatus according to an embodiment of this invention;

FIG. 7 is an example of a flow chart of operation of an inspection apparatus according to an embodiment of this invention;

FIG. 8 is another example of a flow chart of an inspection method according to an embodiment of this invention;

FIG. 9 is another example of a screen structure of an inspection apparatus according to an embodiment of this invention;

FIG. 10 is another example of a flow chart of operation of an inspection apparatus according to an embodiment of this invention;

FIG. 11 is another example of a flow chart of an inspection method according to an embodiment of this invention;

FIG. 12 is another example of a screen structure of an inspection method according to an embodiment of this invention;

FIG. 13 is another example of a flow chart of operation of an inspection apparatus according to an embodiment of this invention;

FIG. 14 is another example of a flow chart of an inspection method according to an embodiment of this invention;

FIG. 15 is another example of a screen structure of an inspection apparatus according to an embodiment of this invention;

FIG. 16 is another example of a flow chart of operation of an inspection apparatus according to an embodiment of this invention;

FIG. 17 is another example of a flow chart of the inspection method according to an embodiment of this invention;

FIG. 18 is another example of a screen structure of an inspection apparatus according to an embodiment of this invention;

FIG. 19 is another example of a flow chart of operation of an inspection apparatus according to an embodiment of this invention;

FIG. 20 is another example of a screen structure of an inspection apparatus according to an embodiment of this invention;

FIG. 21 is another example of a flow chart of operation of an inspection apparatus according to an embodiment of this invention;

FIG. 22 shows an automatic area extraction algorithm to extract the bone marrow of the cranial bones according to an embodiment of this invention;

FIG. 23 is an example of how to define threshold TH according to an embodiment of this invention; and

FIG. 24 shows an automatic area extraction algorithm to extract the bone marrow of the ulna and radius according to an embodiment the invention.

### DETAILED DESCRIPTION OF THE PREFERRED EMBODIMENTS

Next, embodiments of the invention will be described with reference to the drawings.

### First Embodiment

As a predictive phenomenon of the bone density reduction, it is reported that various abnormalities occur in hemopoietic cells (T. Masuzawa, et al., “The Journal of Clinical Investigation 94, 1090-1097 (1994)”). A mouse from which ovarium is extracted and estrogen decreased, B lymphocytes increase specifically in the hemopoietic cells in hemopoietic tissues, while other myeloid cells and granulocytes (neutrophilic leukocytes, eosinophilic leukocytes, basocytes) decrease. Subsequently, the bone resorption increases and the bone density decreases. It is known well that a female hormone, estrogen has effects of facilitating the bone metabolism, proliferating and growing the bone, and suppressing the bone resorption. If the abnormalities in hemopoietic cells precede the bone density reduction caused by decreased estrogen, the bone density reduction may be predicted by detecting these abnormalities in hemopoietic cells.

In general, compared with other hemopoietic cells (comparing with the granulocytes containing many granulations (retentive agents)), lymphocytes are small. The diameters of an eosinophilic leukocyte, a neutrophilic leukocyte, and a basocyte are 13-20 μm, 12-15 μm, and 10-16 μm, respectively, while that of a lymphocyte is 7-10 μm. Based on this fact, if the rate of the large-sized hemopoietic cells decreases, the signal in the bone marrow area containing hemopoietic cells may fall and this can be observed in a nuclear magnetic resonance image of the bone marrow area.

The bone marrow is a soft tissue which fills cavitas medullaris and bony internal substantia spongiosa inside of the bone. Two types of the bone marrow, red marrow and yellow marrow are present. The red marrow consists of a hemopoietic tissue and a fetus or a neonate has only the red marrow. With the growth of a neonate, the hemopoietic cells are replaced by fat, and the red marrow turns into the yellow marrow. However, the flat bone or the long bone epiphysis remains to be red marrow lifelong. The flat bones include cranial bones, a sternum, a costa, a scapula and an ilium, and the long bones include a humerus and a femur. The edge of the long bone is called an epiphysis, an axis thereof is called a diaphysis, and an epiphysis cartilage is present therebetween.

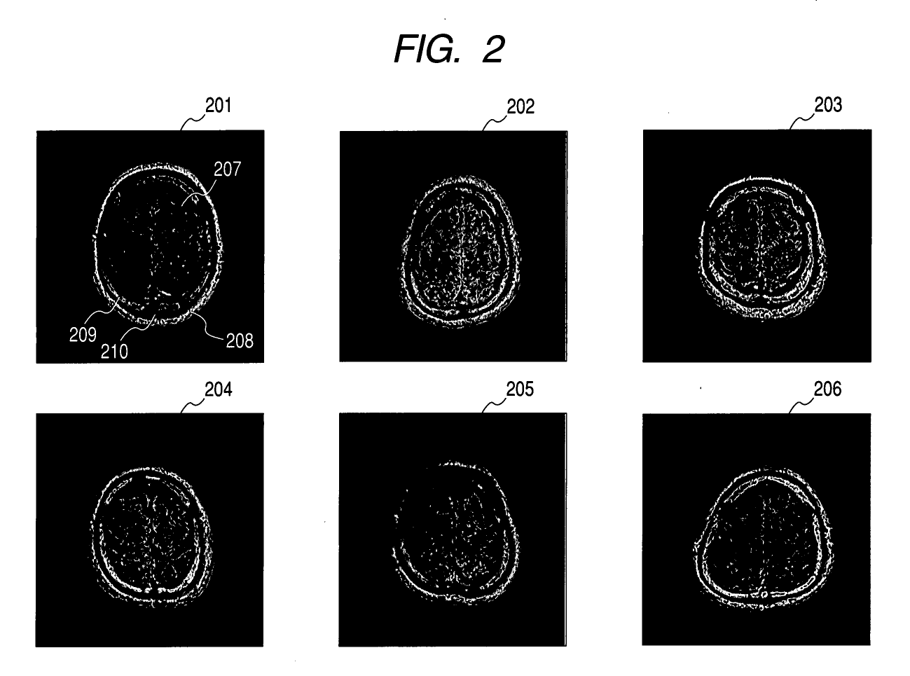

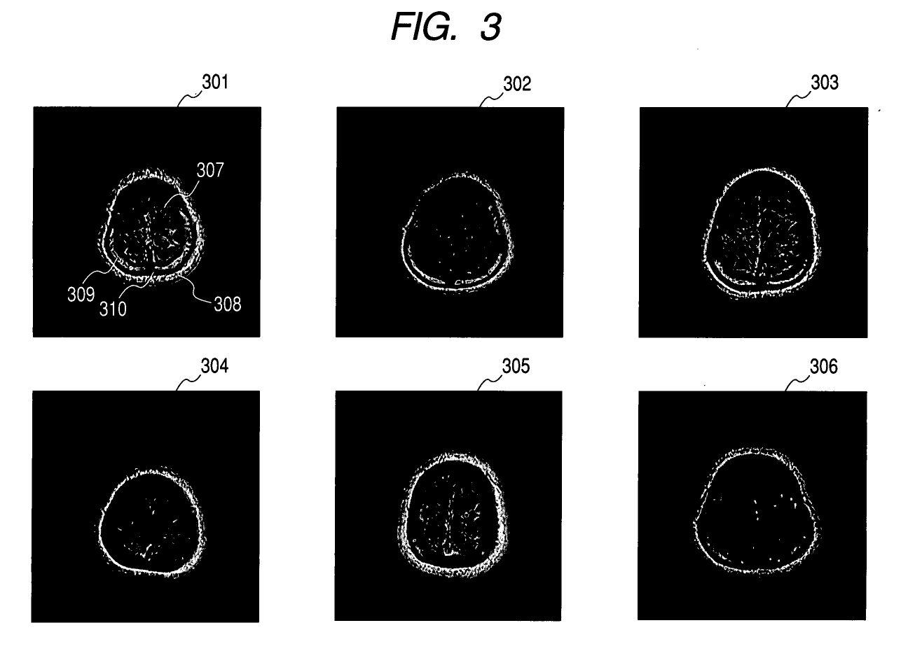

Examples of nuclear magnetic resonance images of the head of males and females in different age brackets are shown in FIGS. 2 and 3. The signal intensity of a nuclear magnetic resonance image reflects not only the density of protons but also transverse relaxation time T2 and longitudinal relaxation time T1. By selecting appropriate measurement parameters, the effects of relaxation times are suppressed and an image which approximately reflects the proton density (proton density image) can be obtained. In order to obtain this image, (1) the repetition time of excitation by RF pulse should be sufficiently long, (2) the interval of signal measurement and excitation should be sufficiently short, and (3) the flip angle of the excitation should be narrow.

Images in FIG. 2 are proton density images of males, wherein 201 to 205 are those of males in their late twenties to early thirties, while 206 is that of a male in his late forties. 207 shows the brain, 208 shows the scalp, 209 shows the bone marrow, and 210 shows bones other than the bone marrow. In the case of a male, no significant difference between different ages can be identified in the contrast of the signal intensity in the bone marrow area and other areas (for example, the scalp, the brain, etc.). Images in FIG. 3 are proton density images of females, wherein 301 to 303 are those of females in their early thirties, while 304 to 306 are those of females in their late thirties to early forties. 307 shows the brain, 308 shows the scalp, 309 shows the bone marrow, and 310 shows bones other than the bone marrow. In the case of a female, the signal in the bone marrow area begins to drop from their late thirties.

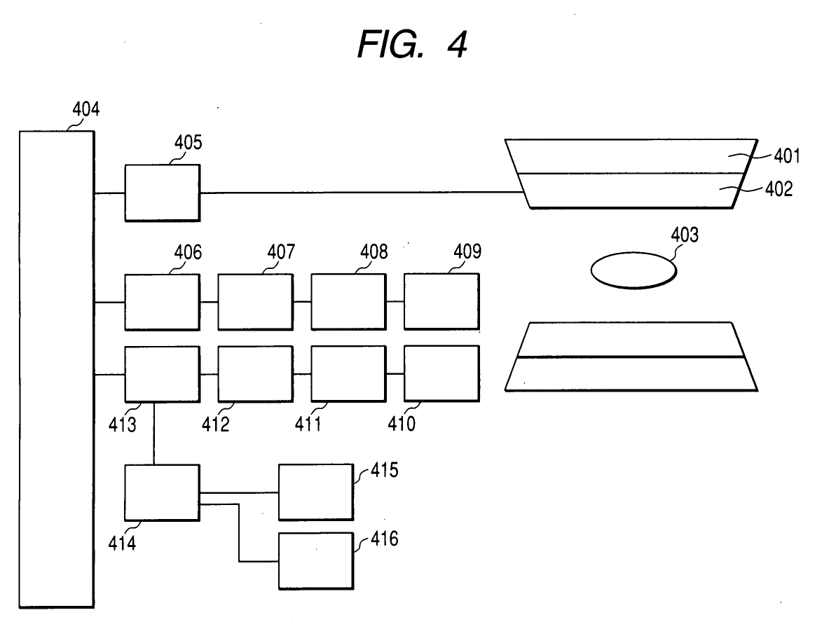

The construction of the inspection apparatus using magnetic resonance of the invention will be described with reference to FIG. 4. In the figure, 401 is a coil for generating gradient magnetic field, 402 is a coil for generating static magnetic field, and 403 is an object. The object is placed in the coils 401 and 402. Sequencer 404 sends an instruction to gradient magnetic field power supply 405 and RF transmitter 406, and gradient magnetic field and RF pulse are applied to object 403. RF pulse is applied to object 403 by RF transmitter 409 via RF modulator 407 and RF amplifier 408. MR signal generated from the object is received by receiver 410, and sent to CPU 414 via amplifier 411, phase detector 412, and AD converter 413. CPU 414 performs signal processing such as reconstruction of a nuclear magnetic resonance image, calculation of area extraction, calculation of the ratio of signal intensities, and determination of the risk of the bone density reduction. The nuclear magnetic resonance image, the result of the calculation and measurement conditions can be stored in memory medium 415 if necessary. The result of the signal processing by CPU 414 can be displayed on display 416.

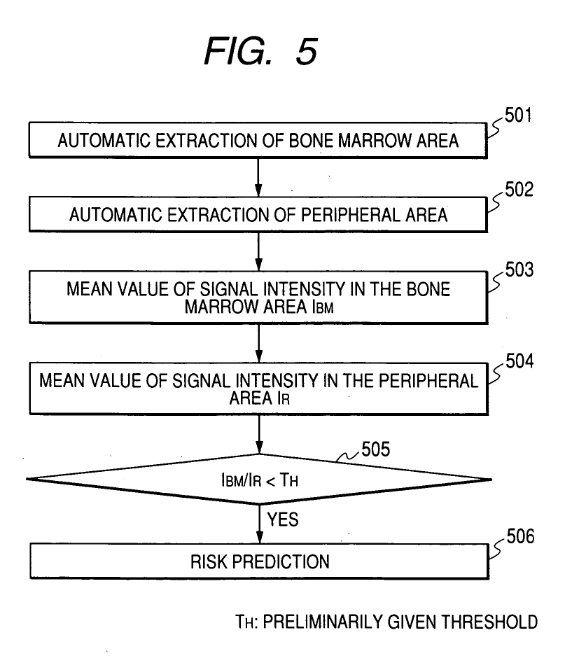

Next, the flow of the inspection method according to the invention will be described with reference to FIG. 5. In this example, a case will be described wherein for the bone marrow of cranial bones, the signal in the peripheral area is selected as a reference signal, and the bone marrow area and the peripheral area are extracted automatically. First, the bone marrow area and the peripheral area are automatically extracted (501, 502), and the mean value of the signal intensity in the bone marrow area IBM and the mean value of the signal intensity in the peripheral area IR are calculated (503, 504). Then ratio of IBM and IR (IBM/IR) is evaluated to determine if it is less than preliminarily given threshold TH (505). When IBM/IR is less than threshold TH, the risk of bone density reduction is predicted (506).

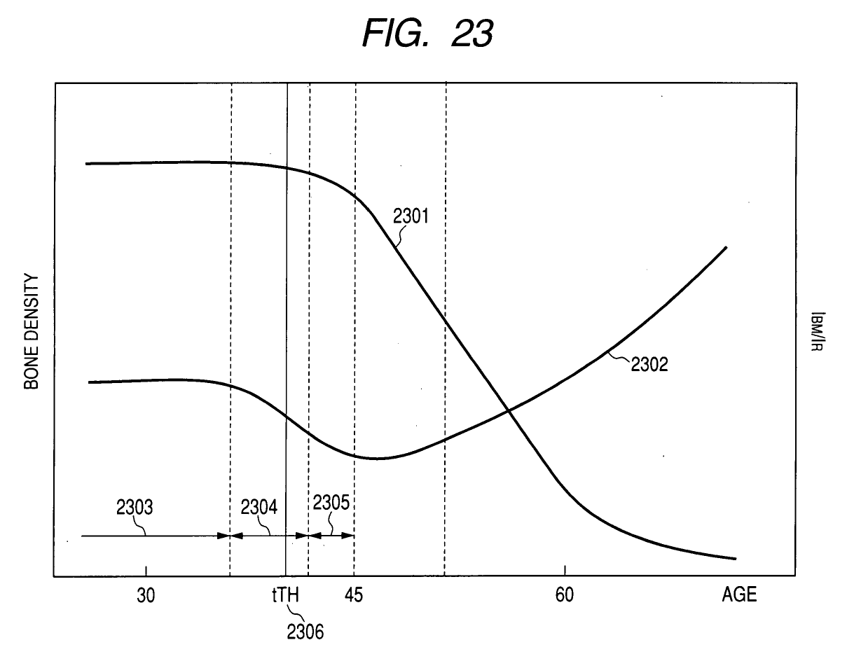

FIG. 23 illustrates an example of how to define threshold TH. The bone density value and IBM/IR of an object are measured several times for many years to calculate a curve 2301 representing the measurements of the bone density and a curve 2302 representing measurements of IBM/IR. One of standard inspection methods such as the DXA method is used for measurement of the bone density. Based on the time when the curve of the measurement of IBM/IR before decline, normal zone 2303 is defined. Based on the time when the curve of the measurement of the bone density begins to decline little by little, and the time when the curve begins abruptly, caution zone 2304 and high risk zone 2305 are defined, respectively. Threshold TH is set to a value of IBM/IR at time tTH (2306) in the range of caution zone 2304 when decrease in the bone density has not begun yet, for example. In order to improve the precision of the threshold, preferably measurement is conducted for many objects.

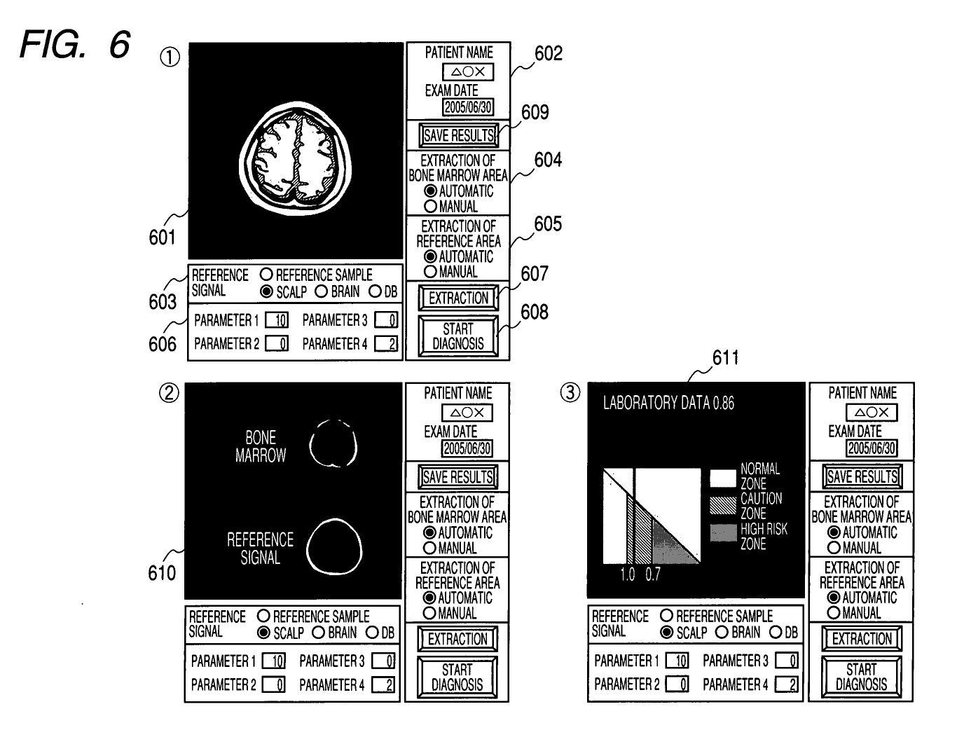

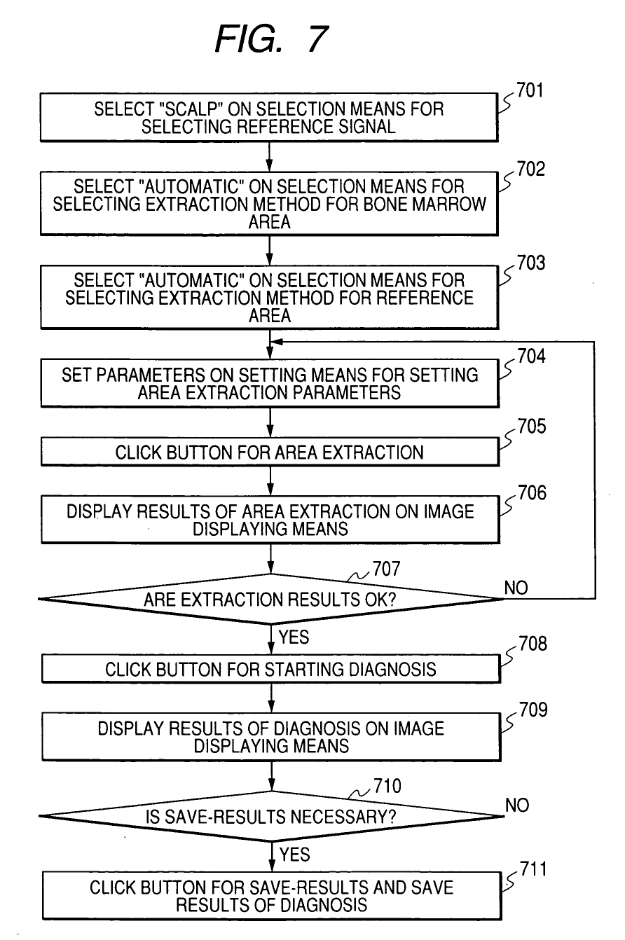

Examples of screen structure and operation flow of the inspection apparatus according to the embodiment will be described with reference to FIGS. 6 and 7. On displaying means for image 601, a nuclear magnetic resonance image of the head containing cranial bones is shown. On displaying means for data property of 602, the name of the patient and exam date is shown. On selecting means for reference signal 603, an area or a database from which a reference signal is extracted is selected. In this embodiment, “scalp” is selected (701). On selection means for selecting method for extracting bone marrow area 604, a method to extract the bone marrow area is selected, i.e., automatic extraction, or manual extraction with use of pointing device. In this example, “automatic” is selected (702). On selection means for selecting method for extracting reference area 605, a method to extract the reference area is selected, i.e., automatic extraction, or manual extraction with use of pointing device. In this example, “automatic” is selected (703). On setting means for setting area extraction parameters 606, parameters used in the automatic extraction of the bone marrow area and the reference area is set (704). The parameters include the type of the nuclear magnetic resonance image used for the area extraction (T1 weighted image, T2 weighted image, proton density image, etc.). Since the signal intensity of a nuclear magnetic resonance image depends on the density of protons and other factors including the longitudinal relaxation time T1 and the transverse relaxation time T2, the contrast among tissues can be modified by adjusting measurement conditions. An image in which the contrast by the difference between T1 is enhanced is called a T1 weighted image, an image in which the contrast by the difference between T2 is enhanced is called a T2 weighted image, and an image which reflects the density of protons by suppressing the effect of relaxation time is called a proton density image.

The parameters include thresholds for separating different tissues to be extracted. When defining a threshold, a histogram of signals is commonly used. If the nuclear magnetic resonance image to be extracted has a sufficient contrast among the tissues, a plurality of peaks that reflect different signals from different tissues will appear on the histogram. Therefore, the threshold may be a signal intensity that separates the peaks. The threshold itself can be input as a parameter, or the number of the peak to be extracted can be specified. Alternatively, the numbers of two peaks to be separated from each other can be specified. The parameters also contain conditions used in the region growing method. The conditions include the upper limit and the lower limit of the area to be extracted. The target area of the region growing method and other area are also specified. After setting the parameters, button for area extraction 607 (705) is clicked. The extraction of the bone marrow area and the reference area is performed automatically, and the result of the extraction is displayed on window for displaying area extraction results 610 of displaying means for image (706). On this window, whether both areas have been extracted satisfactorily can be confirmed (707). In the case of any problem in the extraction result, parameters can be reset in setting means for setting area extraction parameters 606. Then button for area extraction 607 is clicked again to perform automatic extraction of the bone marrow area and the reference area. In the case of no problem in the extraction result, button for starting diagnosis 608 is clicked (708) to determine result of the diagnosis. The diagnosis result is displayed on window for displaying diagnosis results 611 of displaying means for image (709). On this screen, whether the diagnosis result should be saved is determined (710). When it should be saved, button for saving results 609 is clicked to save the diagnosis result (711).

In the above description, the scalp is selected as a peripheral area, but peripheral area is not limited to the scalp. Any area in the brain may be selected as well.

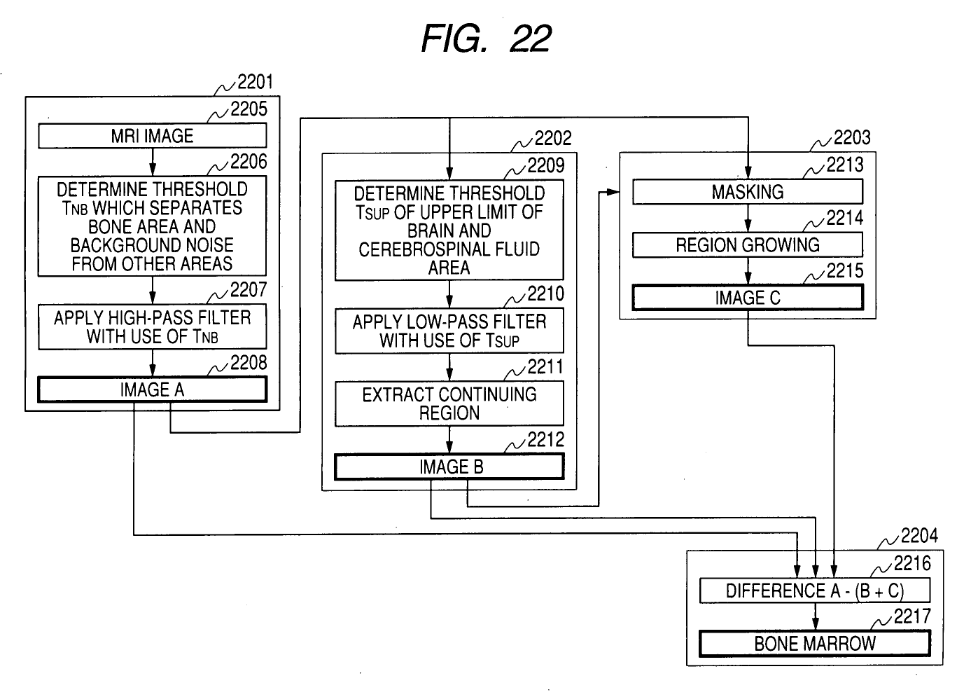

The algorithm used in the automatic area extraction according to the invention will be described with reference to FIG. 22. In 2201, 2202, 2203, 2204 of FIG. 22, segmentation of the brain and the cerebrospinal fluid and the bone marrow, segmentation of the brain and the cerebrospinal fluid, segmentation of the scalp, and segmentation of the bone marrow are performed. In 2205, a nuclear magnetic resonance image is obtained. Since the bone other than the bone marrow is solid, its signal intensity is very low. Threshold TNB which separates the bone and background noise from other areas is determined (2206). Threshold TNB is determined automatically or manually from the histogram representing the signal intensity of MRI image 2205. By applying a high-pass filter to magnetic resonance image 2205 with use of threshold TNB (2207), the background noise and the signal in the bone is eliminated to obtain image A comprising the brain, the cerebrospinal fluid, the scalp, and the bone marrow area (2208). Next, threshold TSUP of the upper limit of the brain and the cerebrospinal fluid area is determined (2209). Threshold TSUP is determined automatically or manually from the histogram representing the signal intensity of image A, for example. A low-pass filter is applied to image A with use of threshold TSUP (2210). From the resultant image, maximum continuous area is extracted to obtain image B comprising the brain and the cerebrospinal fluid. Next, image A is masked by image B (2213) and region growing method is applied to the resultant image (2214), to obtain image C which only comprises the scalp (2215). The region growing method is a technique to extract whole region of interest. In this method, a starting point is defined, then as to a neighboring pixel of the starting point, it is determined if the neighboring pixels satisfies a predetermined condition. When the pixel satisfies the condition, it is determined to belong to a same region. By repeating this step, the whole region of interest is extracted. An example of the condition is that the signal value in a neighboring pixel is within a range defined by the predetermined upper threshold and lower threshold of the tissue area to be extracted. Finally, images B and C are subtracted from image A (2216) to obtain an image of the bone marrow (2217).

### Second Embodiment

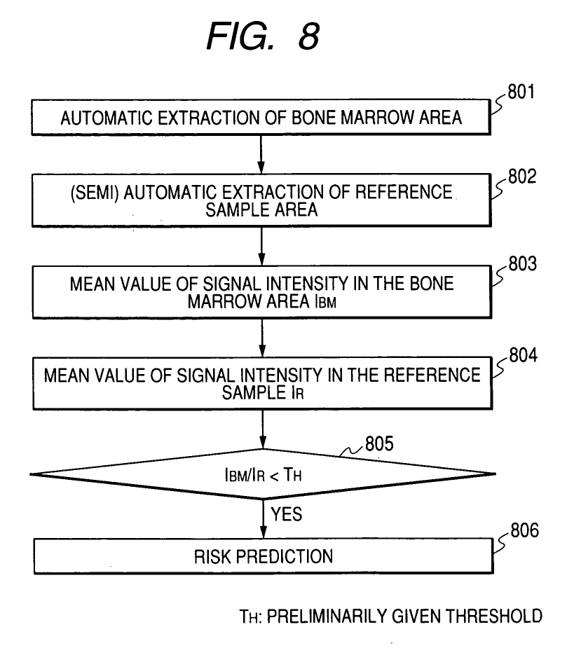

In this embodiment, a case will be described wherein for the bone marrow of the cranial bones, reference sample is used to extract a reference signal, the bone marrow area is extracted automatically, and the reference sample is selected automatically or semi-automatically. For a reference sample, solution of sodium chloride, cupric sulfate, manganese chloride for example, is used. Alternatively, gel of polyvinyl alcohol, or capsule of vitamin E or vitamin D is applicable. Since the structure of the inspection apparatus, how to define threshold TH, the algorithm used in the automatic extraction of the bone marrow of the cranial bones are similar to that described for the embodiment 1, points different from embodiment 1 will be described as follows. First, the flow of the inspection method according to the invention will be described with reference to FIG. 8. The bone marrow area is automatically extracted (801), then the reference sample area is automatically or semi-automatically extracted (802). The mean value of signal intensity in the bone marrow area IBM, and the mean value of signal intensity in the reference sample IR are calculated (803, 804). Then whether the ratio (IBM/IR) between IBM and IR is less than preliminarily given threshold TH is evaluated (805). When IBM/IP is less than threshold TH, risk of bone density reduction can be predicted (806). Although in the above description of the flow of inspection method, the risk is predicted based on the ratio between IBM and IR, the difference between IBM and IR may be compared with the threshold. However, it is difficult to compare absolute values of the nuclear magnetic resonance signals because variation in equipment gain is inevitable over time. Therefore, it is desirable to use the difference between IBM′ and IR′ which can be obtained by dividing IBM and IR by IR, respectively, instead of using values of IBM and IR without change.

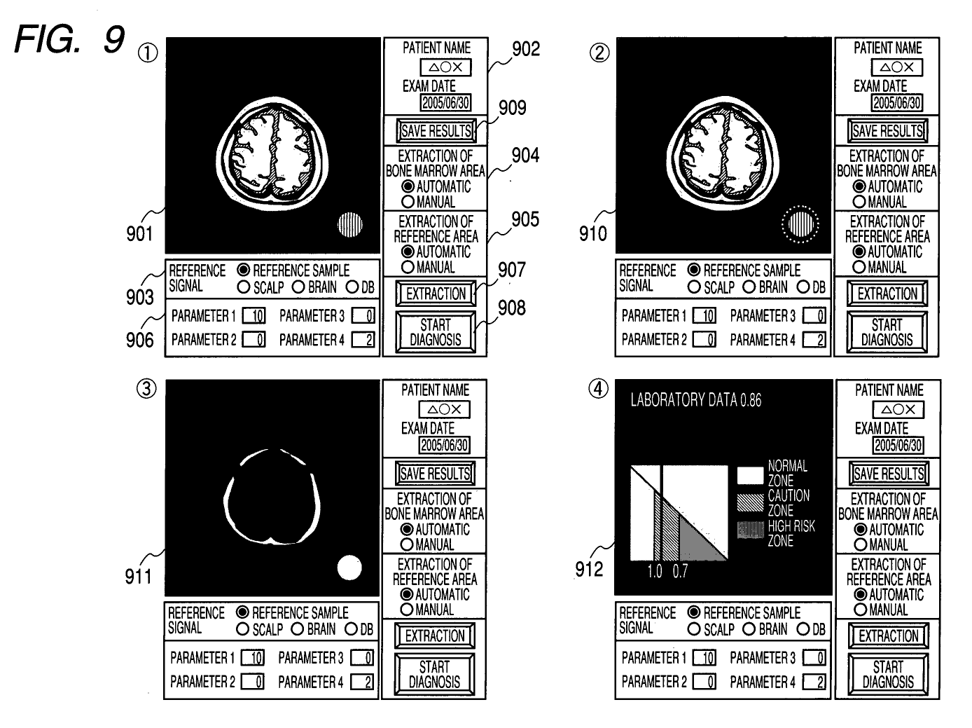

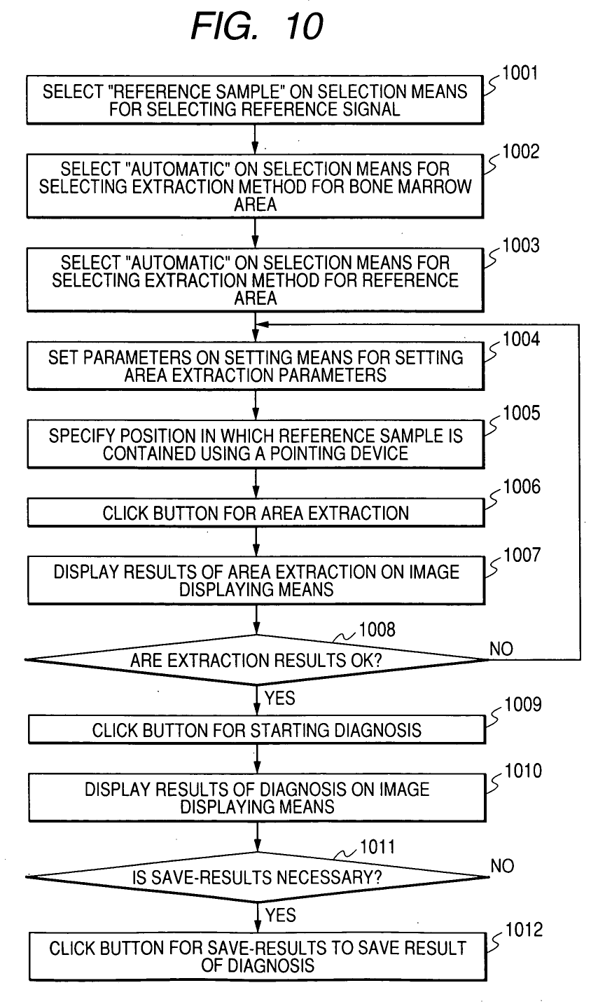

Example of a screen structure and operation flow of the inspection apparatus according to this embodiment will be described with reference to FIGS. 9 and 10. On displaying means for image 801, a nuclear magnetic resonance image of the head containing the cranial bones is displayed. On displaying means for data property 902, the name of patient and exam date is displayed. On selecting means for reference signal 903, an area or a database from which a reference signal is extracted is selected. In this embodiment, “reference sample” is selected (1001). On selection means for selecting method for extracting bone marrow area 904, whether the extraction of the bone marrow area is performed automatically or manually with use of a pointing device is selected. In this example, “automatic” is selected (1002). On selection means for selecting method for extracting the reference area 905, whether the extraction of the reference area is performed automatically or manually with use of a pointing device is selected. In this example, “automatic” is selected (1003). However, completely automatic extraction of an area is difficult unless approximate position of the reference sample on the nuclear magnetic resonance image is known in advance. Automatic extraction may be possible by fixing the position of the reference sample at the time of image capturing and defining the approximate position of the reference sample on a nuclear magnetic resonance image (upper right corner of the image, for example), however, in this embodiment, a case of semi-automatic extraction will be described. In semi-automatic extraction, in a first step, the approximate position of the reference sample is specified with use of a pointing device. The specified approximate position can be rectangular area or circular area containing the reference sample, or a point in the reference sample area. Alternatively, with use of an approximate position specification window, desirable position can be selected from several options such as “upper right corner”, “lower right corner”, “upper left corner”, and “lower left corner”. In a second step, the reference sample area is automatically extracted in a local area which is sufficiently larger than the reference sample area and centered on the approximate position (for example, rectangular area or circular area). In this automatic extraction, the reference sample area is extracted by applying high-pass filter with use of a threshold which separates background noises and the signal area. When “manual” is selected on selection means for selecting method for extracting reference area 905, an area which includes only the reference sample area is specified with use of a pointing device. On setting means for setting area extraction parameters 906, the parameters used in the automatic extraction of the bone marrow area or the reference area are set (1004). Automatic extraction of the sample area by a simple process using a threshold may be possible by defining the approximate position (e.g. lower right corner of the screen) of the reference sample at the time of capturing a nuclear magnetic resonance image, however, in this embodiment, the reference area is semi-automatically extracted by specifying a position containing the reference sample with use of a pointing device on reference sample position specifying screen 810. After setting the parameters, button for area extraction 907 (1006) is clicked again to perform automatic extraction (semi-automatic extraction) of the bone marrow area and the reference area. The extraction result is displayed on window for displaying area extraction results 911 of displaying means for image (1007), thus, whether both areas have been extracted satisfactorily can be confirmed (1008). In the case of any problem in the extraction result, parameters can be reset in setting means for setting area extraction parameters 906. Then button for area extraction 907 is clicked again to perform automatic extraction of the bone marrow area and the reference area. The parameters include the type of the nuclear magnetic resonance image used for the area extraction (T1 weighted image, T2 weighted image, proton density image, etc.). Since the signal intensity of a nuclear magnetic resonance image depends on the density of protons and other factors including the longitudinal relaxation time T1 and the transverse relaxation time T2, the contrast among tissues can be modified by adjusting measurement conditions. An image in which the contrast by the difference between T1 is enhanced is called a T1 weighted image, an image in which the contrast by the difference between T2 is enhanced is called a T2 weighted image, and an image which reflects the density of protons by suppressing the effect of relaxation is called a proton density image. The parameters include thresholds for separating different tissues to be extracted. When defining a threshold, a histogram of signals is commonly used. If the nuclear magnetic resonance image to be extracted has a sufficient contrast among the tissues, a plurality of peaks that reflect different signals from different tissues will appear on the histogram. Therefore, the threshold may be a signal intensity that separates the peaks. The threshold itself can be input as a parameter, or the number of the peak to be extracted in the histogram can be specified. Alternatively, the numbers of two peaks to be separated from each other can be specified. The parameters also include conditions used in the region growing method. The conditions include the upper limit and the lower limit of the area to be extracted. The target area of the region growing method and other area are also specified. The parameters include approximate position of the reference sample (e.g., “upper right corner”, “lower right corner”, “upper left corner”, “lower left corner”). Alternatively, when arbitrary area is specified with use of a pointing device, the parameters include the shape of the area (e.g., rectangle, circle, ellipse). Alternatively, when arbitrary area is specified without use of a pointing device, center of the coordinate of the area, size or shape of the area can be specified in a text. In the case of no problem in the extraction result, button for starting diagnosis 908 (1009) is clicked to determine result of the diagnosis. The diagnosis result is displayed on window for displaying diagnosis results 812 of the displaying means for image (1010). On this screen, whether the diagnosis result should be saved is determined (1011). When it should be saved, button for saving results 909 is clicked to save the diagnosis result (1012).

In the above description, the ratio between IBM and IR is compared with TH. However, similar evaluation can be performed by converting IBM and IR to absolute values with use of absolute concentration of the reference sample, and comparing them with other threshold. Furthermore, the scalp is selected as a peripheral area in the above description, but peripheral area is not limited to the scalp. Other areas in the brain may be selected as well.

### Third Embodiment

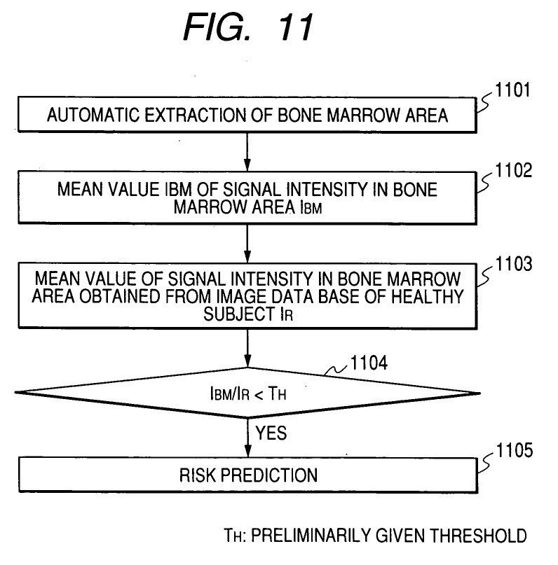

In this embodiment, a case will be described wherein for the bone marrow of the cranial bones, a signal in the bone marrow of a healthy control stored in a database is selected as a reference signal, and the bone marrow area is extracted automatically. Since the structure of the inspection apparatus, how to define threshold TH, the algorithm used in the automatic extraction of the bone marrow of cranial bones are similar to that described for the embodiment 1, points different from embodiment 1 will be described as follows. First, the flow of inspection method according to the invention will be described with reference to FIG. 11. The bone marrow area is automatically extracted to calculate mean value IBM of the signal intensity in the bone marrow area (1102). Mean value IR of the signal intensity in the bone marrow area is calculated from an image database of healthy controls (1103). Then whether the ratio (IBM/IR) of IBM and IR is less than preliminarily given threshold TH is evaluated (1104). When IBM/IP is less than threshold TH, risk of bone density reduction can be predicted (1105).

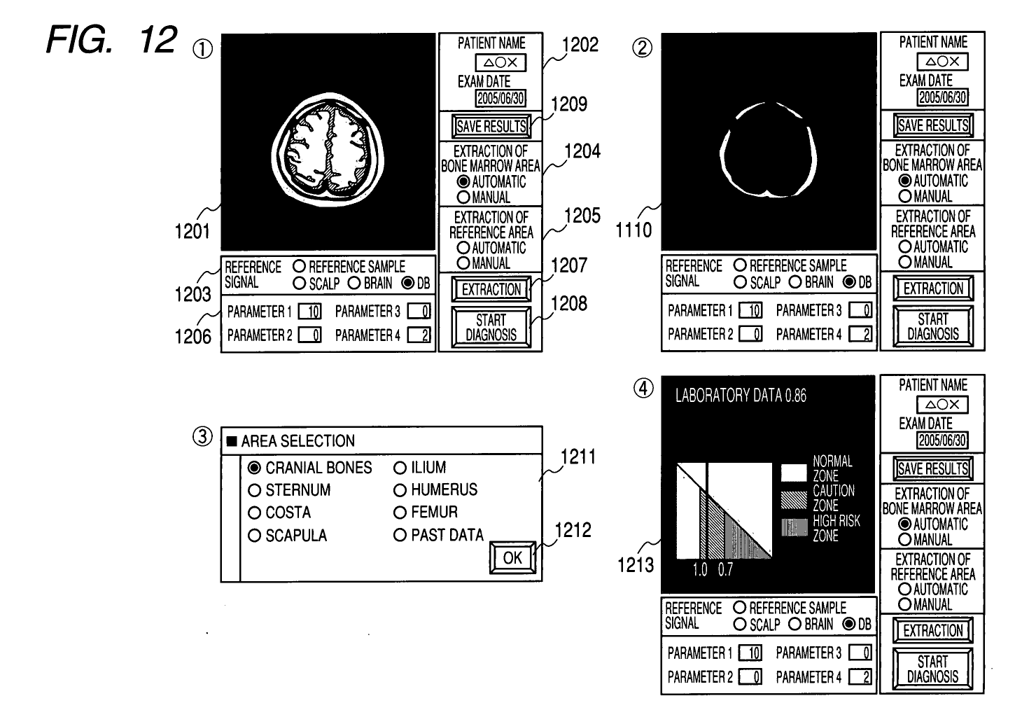

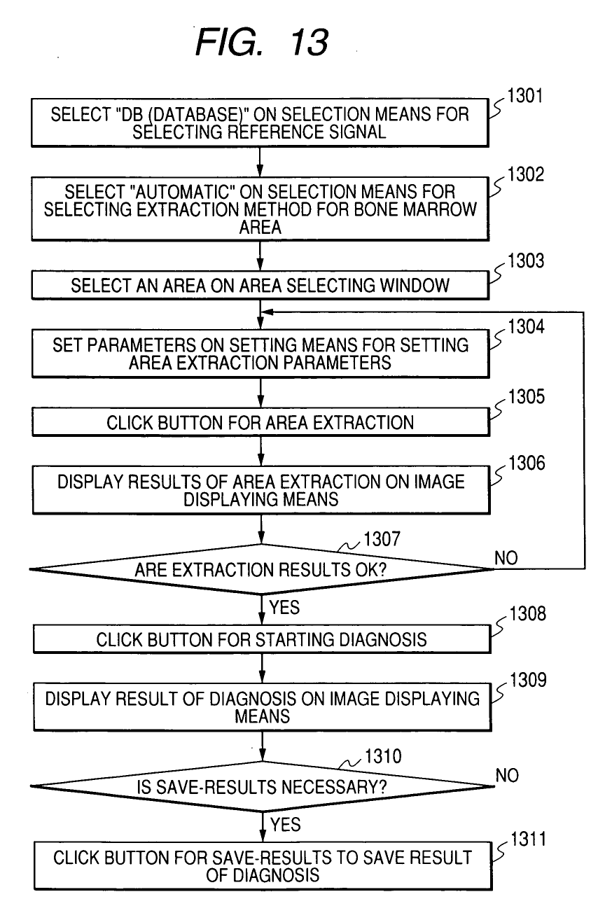

Examples of a screen structure and operation flow of the inspection apparatus according to this embodiment will be described with reference to FIGS. 12 and 13. In displaying means for image 1201, a nuclear magnetic resonance image of the head containing the cranial bones is displayed. On displaying means for data property 1202, name of the patient and exam date are displayed. On selecting means for reference signal 1203, an area or database from which a reference signal is extracted is selected. In this embodiment, “DB (database)” is selected (1301). On selection means for selecting method for extracting bone marrow area 1104, whether the extraction of bone marrow area is performed automatically or manually with use of a pointing device is selected. In this example, “automatic” is selected (1302). On selection means for selecting method for extracting reference area 1205, whether the extraction of the reference area is performed automatically or manually with use of a pointing device is selected. Nothing is selected in this embodiment. On selection window for selecting area 1211, the site of the bone marrow from which a reference signal is extracted is selected (1303), and determined by clicking OK button 1212. “Cranial bones” is selected in this embodiment. On setting means for setting area extraction parameters 1206, parameters used in the automatic extraction of a bone marrow area is set (1304). The parameters include the type of the nuclear magnetic resonance image used for the area extraction (T1 weighted image, T2 weighted image, proton density image, etc.). Since the signal intensity of a nuclear magnetic resonance image depends on the density of protons and other factors including the longitudinal relaxation time T1 and the transverse relaxation time T2, the contrast among tissues can be modified by adjusting measurement conditions. An image in which the contrast by the difference between T1 is enhanced is called a T1 weighted image, an image in which the contrast by the difference between T2 is enhanced is called a T2 weighted image, and an image which reflects the density of protons by reducing the effect of relaxation is called a proton density image. The parameters include a threshold for separating different tissues to be extracted. When defining a threshold, a histogram of a signal is commonly used. If the nuclear magnetic resonance image to be extracted has a sufficient contrast among the tissues, a plurality of peaks that reflect different signals from different tissues will appear on the histogram. Therefore, the threshold may be a signal intensity that separates the peaks. The threshold itself can be input as a parameter, or the number of the peak to be extracted in the histogram can be specified. Alternatively, the numbers of two peaks to be separated from each other can be specified. The parameters also contain conditions used in the region growing method. The conditions include the upper limit and the lower limit of the area to be extracted. The target area of the region growing method and other area are also specified. After setting parameters, button for area extraction 1207 (1305) is clicked to perform automatic extraction of the bone marrow area. The extraction result is displayed on window for displaying area extraction results 1210 of displaying means for image (1306) thus, whether both areas have been extracted satisfactorily can be confirmed (1307). In the case of any problem in the extraction result, parameters can be reset in setting means for setting area extraction parameters 1206. Then button for area extraction 1207 is clicked again to perform automatic extraction of the bone marrow area and the reference area. In the case of no problem in the extraction result, button for starting diagnosis 1208 (1308) is clicked to determine result of the diagnosis. The diagnosis result is displayed on window for displaying diagnosis results 1213 of the displaying means for image (1309). On this screen, whether the diagnosis result should be saved is determined (1310). When it should be saved, button for saving results 1209 is clicked to save the diagnosis result (1311).

In the above description, the scalp is selected as a peripheral area, but the peripheral area is not limited to the scalp. Other areas in the brain may be selected as well.

### Fourth Embodiment

In this embodiment, a case will be described wherein for the bone marrow of cranial bones, a signal in a peripheral area is selected as a reference signal, and the bone marrow area and the peripheral area are extracted manually. Since the structure of the inspection apparatus, and how to define threshold TH are similar to that described for the embodiment 1, points different from embodiment 1 will be described as follows.

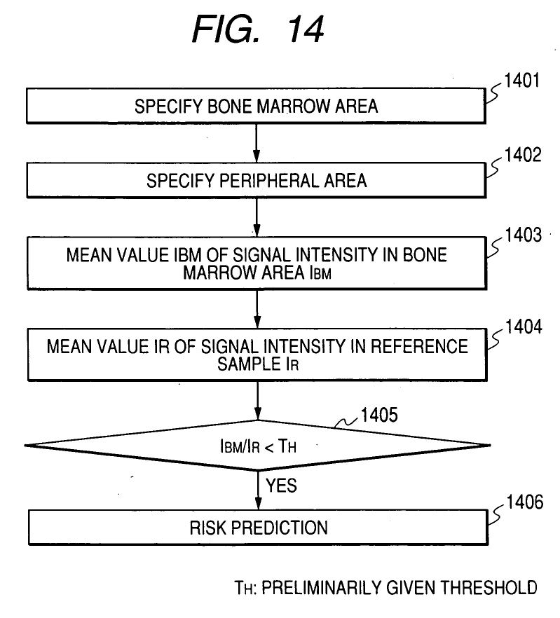

First, the flow of inspection method according to the invention will be described with reference to FIG. 14. The bone marrow area and the peripheral area are specified manually (1401, 1402), and the mean value IBM of signal intensity in the bone marrow area and the mean value IR of signal intensity in the peripheral area are calculated (1403, 1404). Then whether the ratio (IBM/IR) of IBM and IR is less than preliminarily given threshold TH is evaluated (1405). When IBM/IP is less than threshold TH, risk of bone density reduction can be predicted (1406).

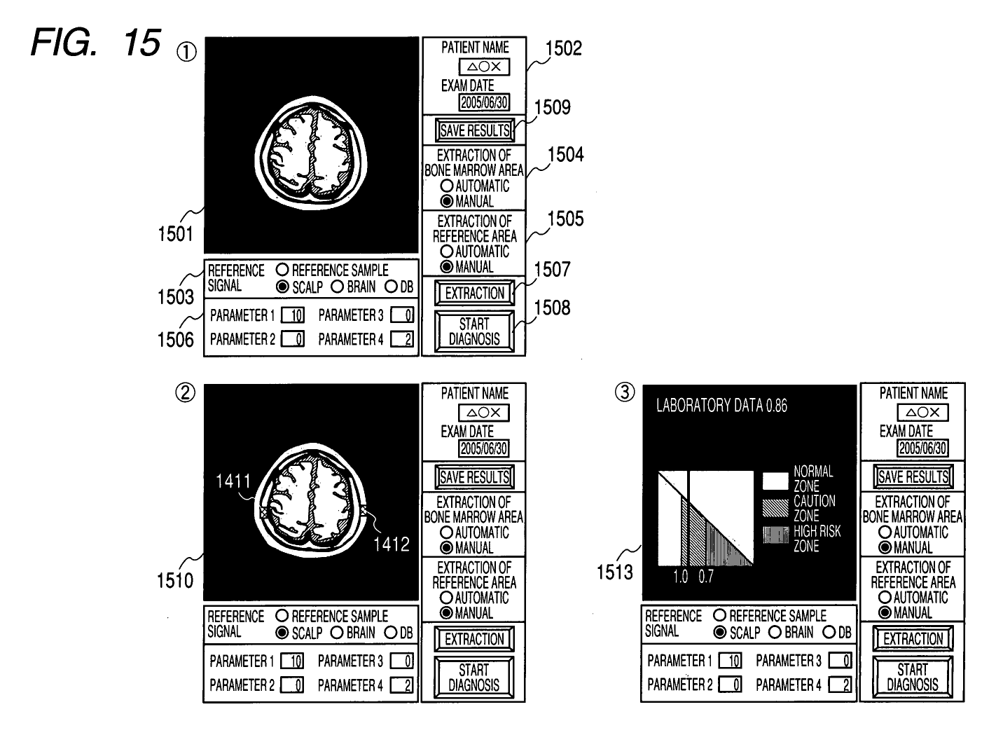

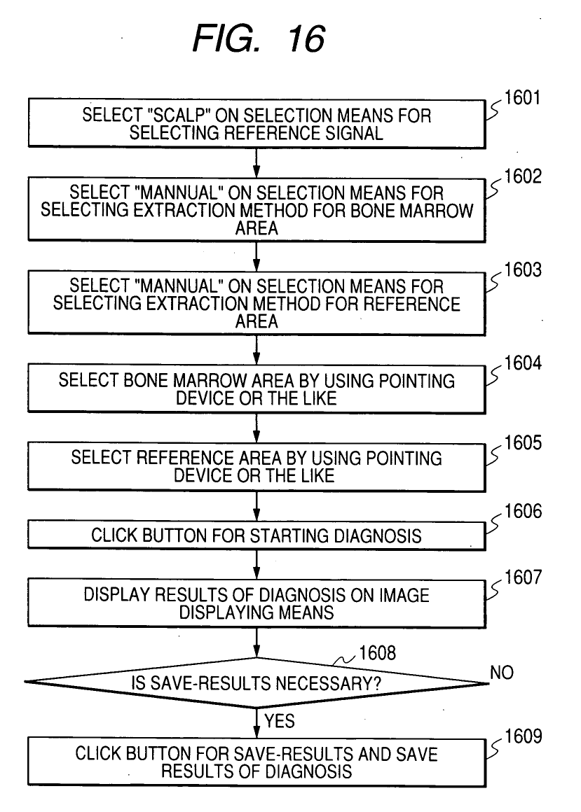

Examples of a screen structure and operation flow of the inspection apparatus according to the invention will be described with reference to FIGS. 15 and 16. On displaying means for image 1401, a nuclear magnetic resonance image of the head containing the cranial bones is displayed. On displaying means for data property 1502, name of the patient and exam date are displayed. On selecting means for reference signal 1503, an area or database from which a reference signal is extracted is selected. In this embodiment, “scalp” is selected (1601). On selection means for selecting method for extracting bone marrow area 1504, whether the extraction of the bone marrow area is performed automatically or manually with use of a pointing device is selected. In this embodiment, “manual” is selected (1602). On selection means for selecting method for extracting reference area 1505, whether the extraction of the reference area is performed automatically or manually with use of a pointing device is selected. In this embodiment, “manual” is selected (1603). On setting means for setting area extraction parameters 1506, parameters to be used in the automatic extraction of the bone marrow area and the reference area are set. In this embodiment, however, as extraction is performed manually, setting means 1506 is not used. On the nuclear magnetic resonance image, bone marrow area 1511 and reference area 1512 are selected using a pointing device (1604, 1605). The selected bone marrow area and reference area are displayed on area extraction result display 1510 of the displaying means for image. After area selection, button for starting diagnosis 1508 is clicked to determine the diagnosis result (1606). The diagnosis result is displayed on window for displaying diagnosis results 1513 of the displaying means for image (1607). On this screen, whether the diagnosis result should be saved is determined (1508). When it should be saved, button for saving results 1409 is clicked to save the diagnosis result (1609).

In the above description, the scalp is selected as a peripheral area, but the peripheral area is not limited to the scalp. Other areas in the brain may be selected as well.

### Fifth Embodiment

In this embodiment, a case will be described wherein for the bone marrow of the cranial bones, signal intensity in the bone marrow area of an object in the past which has been stored in a database is selected as a reference signal, and the bone marrow area is extracted automatically. Since the structure of the inspection apparatus, how to define threshold TH, and the algorithm used in automatic extraction of the bone marrow of cranial bones are similar to that described for the embodiment 1, points different from embodiment 1 will be described as follows.

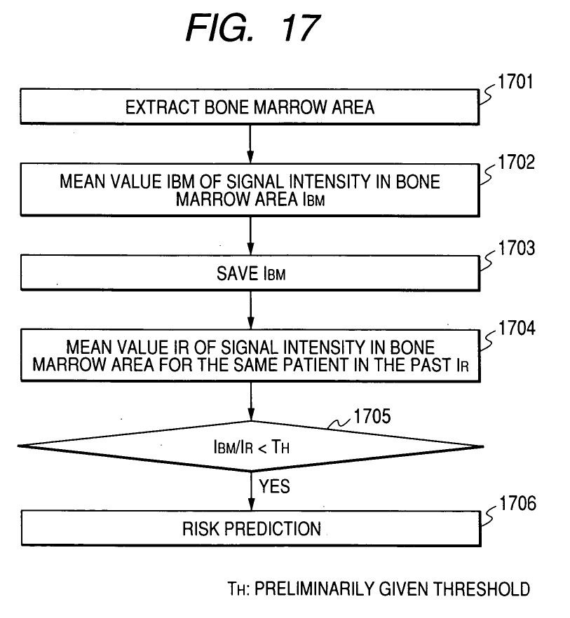

First, the flow of inspection method according to the invention will be described with reference to FIG. 17. The bone marrow area is extracted automatically (1701), mean value IBM of the signal intensity in the bone marrow area is calculated (1702), and IBM is stored in a database (1703). The mean value IR of the signal intensity in the bone marrow which has been obtained from same object in the past is calculated (1704). Then whether the ratio (IBM/IR) of IBM and IR is less than preliminarily given threshold TH is evaluated (1705). When IBM/IP is less than threshold TH, risk of bone density reduction can be predicted (1706).

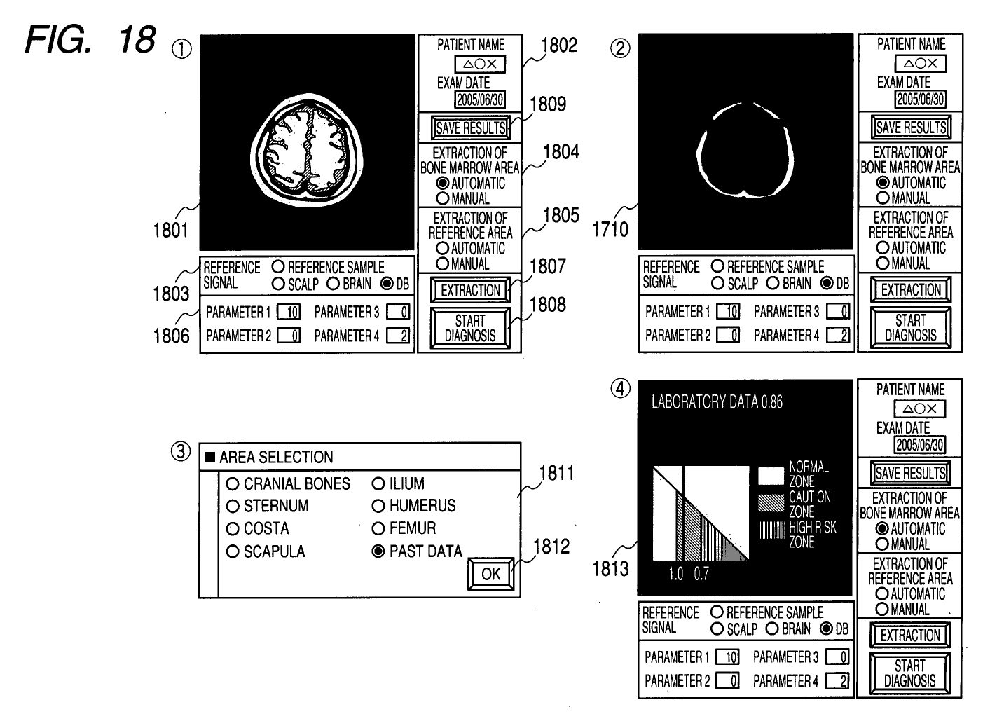

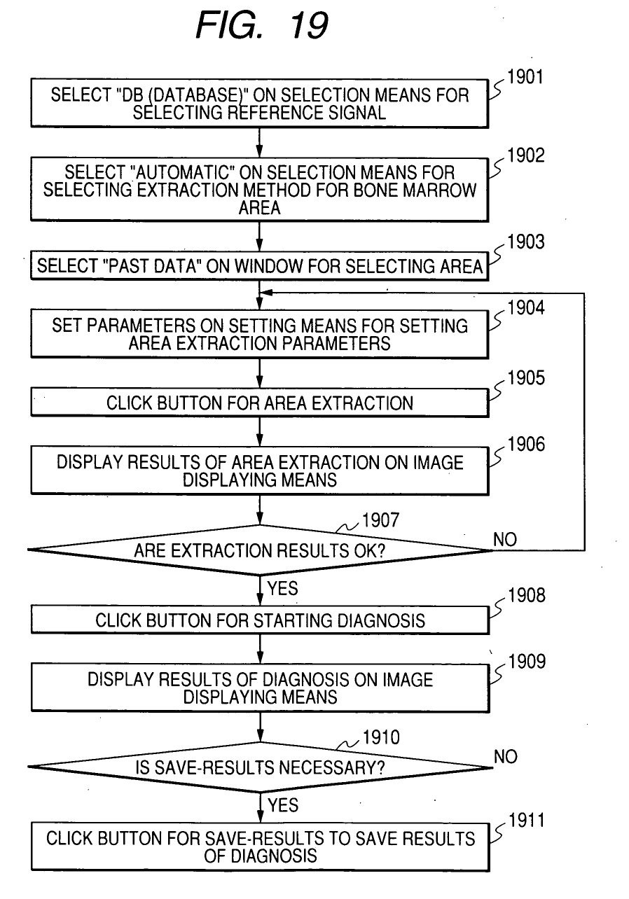

Example of screen structure and operation flow of the inspection apparatus according to this embodiment will be described with reference to FIGS. 18 and 19. On displaying means for image 1801, a nuclear magnetic resonance image of the head containing the cranial bones is displayed. On displaying means for data property 1802, name of the patient and exam date are displayed. On selection means for reference signal 1803, an area or database from which a reference signal is extracted is selected. In this example, “DB (database)” is selected (1901). On selection means for selecting method for extracting bone marrow area 1704, whether the extraction of the bone marrow area is performed automatically or manually with use of a pointing device is selected. In this embodiment, “automatic” is selected (1902). On selection means for selecting method for extracting reference area 1805, whether the extraction of the reference area is performed automatically or manually with use of a pointing device is selected. Nothing is selected in this embodiment. On selection window for selecting area 1811, “past data” is selected (1903) and OK button 1812 is clicked to determine the selection. On setting means for setting area extraction parameters 1806, parameters used for the automatic extraction of the bone marrow area are set (1904). The parameters include the type of the nuclear magnetic resonance image used for the area extraction (T1 weighted image, T2 weighted image, proton density image, etc.). Since the signal intensity of a nuclear magnetic resonance image depends on the density of protons and other factors including the longitudinal relaxation time T1 and the transverse relaxation time T2, the contrast among tissues can be modified by adjusting measurement conditions. An image in which the contrast by the difference between T1 is enhanced is called a T1 weighted image, an image in which the contrast by the difference between T2 is enhanced is called a T2 weighted image, and an image which reflects the proton density by reducing the effect of relaxation time is called a proton density image. The parameters include thresholds for separating different tissues to be extracted. When defining a threshold, a histogram of a signal is commonly used. If the nuclear magnetic resonance image to be extracted has a sufficient contrast among the tissues, a plurality of peaks that reflect different signals from different tissues will appear on the histogram. Therefore, the threshold may be a signal intensity that separates the peaks. The threshold itself can be input as a parameter, or the number of the peak to be extracted can be specified. Alternatively, the numbers of the peaks to be separated from each other in the histogram can be specified. The parameters also contain conditions used in the region growing method. The conditions include the upper limit and the lower limit of the area to be extracted. The target area of the region growing method and other area are also specified. After setting parameters, button for area extraction 1807 (1905) is clicked to perform automatic extraction of the bone marrow area. The extraction result is displayed on window for displaying area extraction results 1710 of the displaying means for image (1906) thus, whether both areas have been extracted satisfactorily can be confirmed (1907). In the case of any problem in the extraction result, parameters can be reset in setting means for setting area extraction parameters 1806. Then button for area extraction 1807 is clicked again to perform automatic extraction of the bone marrow area and the reference area. In the case of no problem in the extraction result, button for starting diagnosis 1808 (1908) is clicked to determine result of the diagnosis. The diagnosis result is displayed on window for displaying diagnosis results 1813 of the displaying means for image (1909). On this screen, whether the diagnosis result should be saved is determined (1910). When it should be saved, button for saving results 1809 is clicked to save the diagnosis result (1911).

In the above description, the scalp is selected as a peripheral area, but the peripheral area is not limited to the scalp. Other areas in the brain may be selected as well.

### Sixth Embodiment

In this embodiment, a case will be described wherein for the ulna and radius, a signal in a peripheral area is selected as a reference signal, and the bone marrow area and the peripheral area are extracted automatically. Since the structure of the inspection apparatus, flow of the inspection method, and how to define threshold TH are similar to that described for the embodiment 1, points different from embodiment 1 will be described as follows.

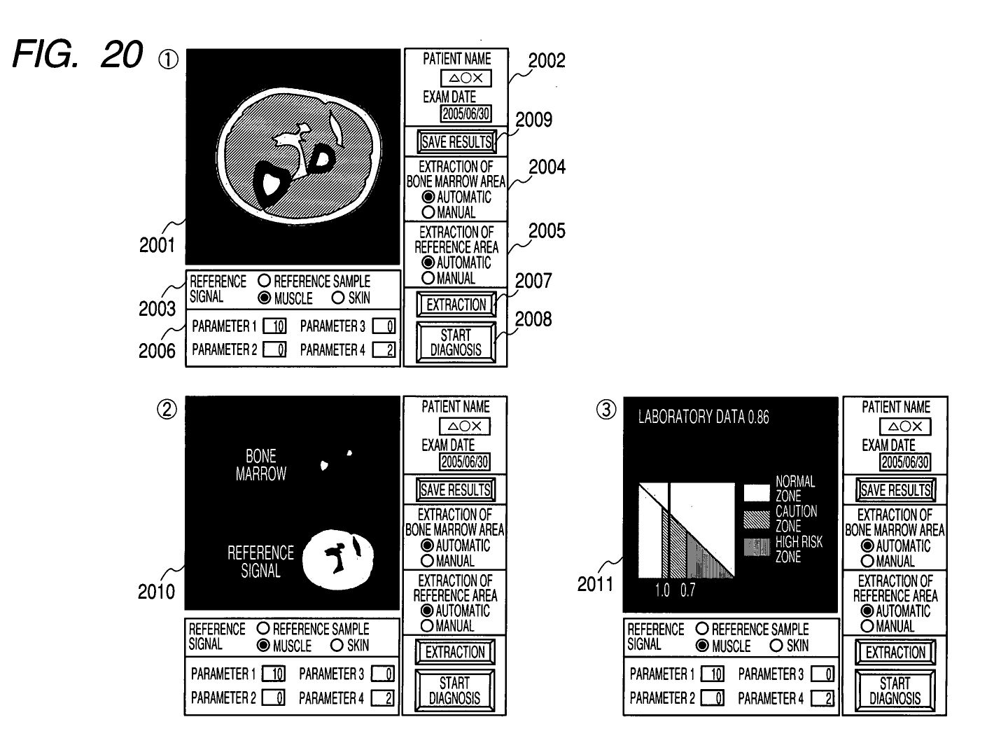

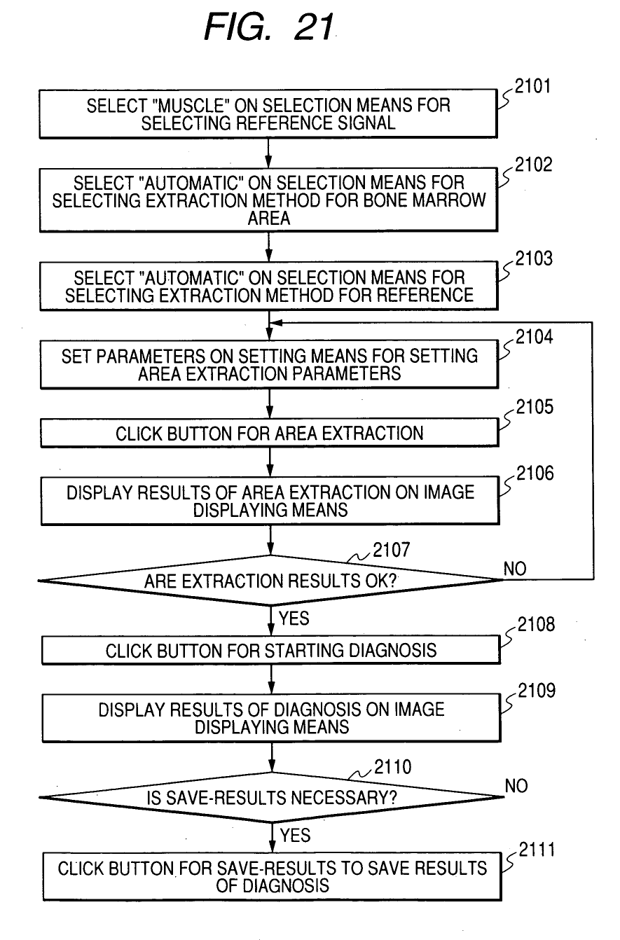

Example of a screen structure and operation flow of the inspection apparatus according to this embodiment will be described with reference to FIGS. 20 and 21. On displaying means for image 2001, a nuclear magnetic resonance image of the head containing the cranial bones is displayed. On displaying means for data property 2002, name of the patient and exam date are displayed. On selecting means for reference signal 2003, the area or database from which a reference signal is extracted is selected. In this example, “muscle” is selected (2101). On selection means for selecting method for extracting bone marrow area 2004, whether the extraction of the bone marrow area is performed automatically or manually with use of a pointing device is selected. In this embodiment, “automatic” is selected (2102). On selection means for selecting method for extracting reference area 2005, whether the extraction of the reference area is performed automatically or manually with use of a pointing device is selected. In this embodiment, “automatic” is selected (2103). On setting means for setting area extraction parameters 2006, parameters used for the automatic extraction of the bone marrow area and the reference area is set (2104). The parameters include the type of the nuclear magnetic resonance image used for the area extraction (T1 weighted image, T2 weighted image, proton density image, etc.). Since the signal intensity of a nuclear magnetic resonance image depends on the density of protons and other factors including the longitudinal relaxation time T1 and the transverse relaxation time T2, the contrast among tissues can be modified by adjusting measurement conditions. An image in which the contrast by the difference between T1 is enhanced is called a T1 weighted image, an image in which the contrast by the difference between T2 is enhanced is called a T2 weighted image, and an image which reflects a proton density by reducing the effect of relaxation time is called a proton density image. The parameters include threshold for separating different tissues to be extracted. When defining a threshold, a histogram of a signal is commonly used. If the nuclear magnetic resonance image to be extracted has a sufficient contrast among the tissues, a plurality of peaks that reflect different signals from different tissues will appear on the histogram. Therefore, the threshold may be a signal intensity that separates the peaks. The threshold itself can be input as a parameter, or the number of the peak to be extracted can be specified. Alternatively, the numbers of the peaks to be separated from each other in the histogram can be specified. The parameters also contain conditions used in the region growing method. The conditions include the upper limit and the lower limit of the area to be extracted. The target area of the region growing method and other area are also specified. After setting parameters, button for area extraction 2007 (2105) is clicked to perform automatic extraction of the bone marrow area and the reference area. The extraction result is displayed on window for displaying area extraction results 2010 of displaying means for image (2106) thus, whether both areas have been extracted satisfactorily can be confirmed (2107). In the case of any problem in the extraction result, parameters can be reset in setting means for setting area extraction parameters 2006. Then button for area extraction 2007 is clicked again to perform automatic extraction of the bone marrow area and the reference area. In the case of no problem in the extraction result, button for starting diagnosis 2008 (2108) is clicked to determine result of the diagnosis. The diagnosis result is displayed on window for displaying diagnosis results 2011 of the displaying means for image (2109). On this screen, whether the diagnosis result should be saved is determined (2110). When it should be saved, button for saving results 2009 is clicked to save the diagnosis result (2111).

In the above description, the muscle is selected as a peripheral area, but the peripheral area is not limited to the muscle. Other area such as skin may be selected as well.

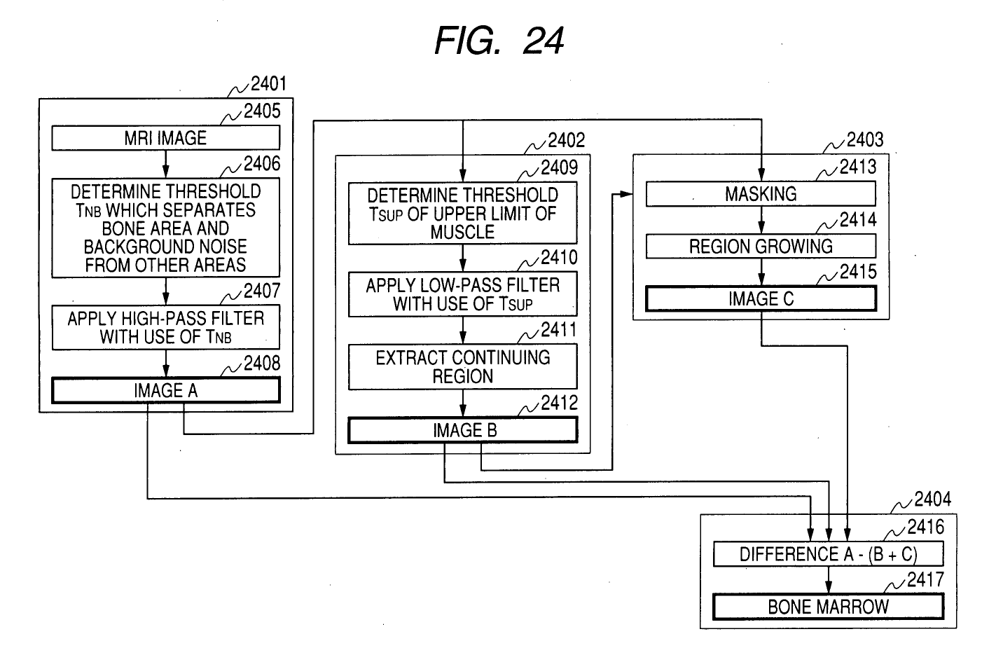

The algorithm used in the automatic bone marrow area extraction of ulna and radius according to the invention will be described with reference to FIG. 24. In 2401, 2402, 2403, 2404 of FIG. 24, segmentation of the muscle, the skin, and the bone marrow, segmentation of the muscle, segmentation of the skin, and segmentation of the bone marrow are performed. In 2405, a nuclear magnetic resonance image (MRI image) is obtained. Since the bone other than the bone marrow is solid, their signal intensity is very low. Threshold TNB which separates the bone and background noise from other areas is determined (2406). Threshold TNB is determined automatically or manually from the histogram of signal intensity of the magnetic resonance image 2205. By applying a high-pass filter to the magnetic resonance image 2205 with use of threshold TNB (2407), the background noise and the signal in the bone is eliminated to obtain image A comprising the muscle, the skin and the bone marrow area (2408). Next, threshold TSUP for the upper limit of the muscle area is determined (2409). Threshold TSUP is determined automatically or manually from the histogram of the signal intensity of image A, for example. A low pass filter is applied to image A with use of threshold TSUP (2410). From the resultant image, maximum continuous area is extracted (2411) to obtain Image B comprising the muscle (2412). Next, image A is masked by image B (2413) and the region growing method is applied to the resultant image (2414) to obtain image C which only comprises the skin (2415). Finally, images B and C are subtracted from image A (2416) to obtain an image of the bone marrow (2417).

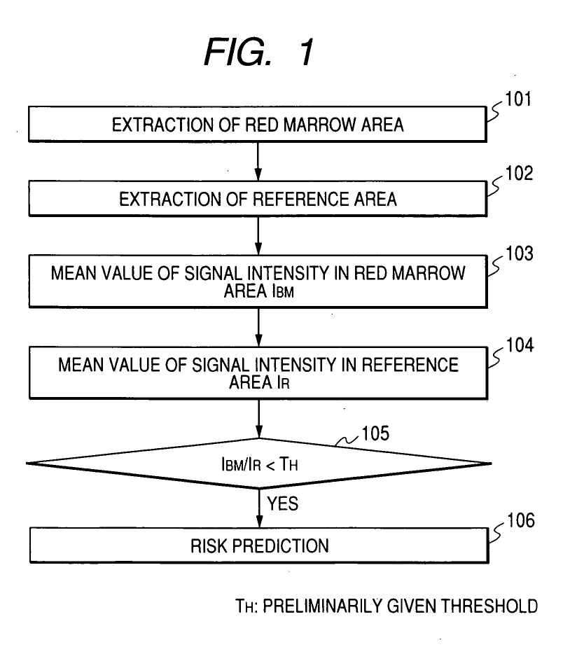

In the above embodiment, the signal of the bone marrow of the cranial bone, the ulna or the radius is used to predict the bone density reduction, however, the area to be used is not limited thereto. Instead, any area which retains red marrow lifelong is applicable. As shown in FIG. 1, in this invention, the risk of bone density reduction is predicted (106) as follows: extracting a red marrow area having hemopoietic cells (101) and area other than the red marrow area as a reference area (102), from a nuclear magnetic resonance image containing a flat bone or a long bone epiphysis, calculating mean value IBM of the signal intensity in the red marrow area (103) and mean value IR of the signal intensity in the reference area (104), and evaluating whether the ratio (IBM/IR) of IBM and IR is less than preliminarily given threshold TH (105). When IBM/IP is less than threshold TH, the risk of bone density reduction is predicted (106). As described in detail, in this invention, by evaluating the signal intensity of the red marrow area, abnormalities in the bone marrow hemopoietic cells can be detected prior to actual reduction in bone density. Thus, the bone density reduction can be predicted before it actually occurs.

As described in detail, in this invention, by obtaining a nuclear magnetic resonance image of a region containing a flat bone or a long bone epiphysis, and evaluating the signal intensity of the red marrow area, abnormalities in the bone marrow hemopoietic cells can be detected before bone density reduction actually occurs. In other words, the bone density reduction can be predicted before it actually occurs. In conventional methods, the red marrow of sternum is extracted by bone marrow puncture to examine the ratio of the hemopoietic cells in the bone marrow. The invention does not use such invasive method. Instead, it can detect abnormalities in hemopoietic cells by evaluating the signal intensity of the bone marrow area in a nuclear magnetic resonance image.

What is claimed is:

1. An inspection apparatus using magnetic resonance comprising:
a static magnetic field generating device which generates static magnetic field in an area where an object is placed;
a gradient magnetic field generating device which gives gradient to the static magnetic field generated by the static magnetic field generating device;
a transmitter which applies radio frequency pulse (RF pulse) to the object;
a receiver which receives nuclear magnetic resonance signals generated from the object;
a control device which controls operation of the static magnetic field generating device, the gradient magnetic field generating device, the transmitter, and the receiver;
an processing device which compares 1) the intensity of the nuclear magnetic resonance signal in the bone marrow area of the flat bone or the long bone epiphysis, with 2) the intensity of the nuclear magnetic resonance signal in an area other than the bone marrow area; and
a display device which displays the result of the calculation of the processing device.

2. An inspection apparatus using magnetic resonance of claim 1, wherein the processing device has an area extraction means which automatically extracts 1) the bone marrow area of the flat bone or the long bone epiphysis, and 2) an area other than the bone marrow area, from the nuclear magnetic resonance signal received by the receiver.

3. An inspection apparatus using magnetic resonance of claim 1, wherein the area extraction means extracts the bone marrow area by performing:
a first process to obtain a first image which comprises the brain area, the cerebrospinal fluid area, the scalp area, and the bone marrow area, by applying a high-pass filter with use of a first threshold to the nuclear magnetic resonance signal from the cranial bones of the object received by the receiver;
a second process to obtain a second image which comprises the brain area and the cerebrospinal fluid area, by applying a low-pass filter with use of a second threshold to the first image and extracting the maximum continuing area therefrom;
a third process to obtain a third image which comprises the scalp areas, by masking the first image by the second image and conducting the region growing method on the resultant image; and
a forth process to obtain a forth image which comprises the bone marrow area, by subtracting the second and third images from the first image to extract the bone marrow areas.

4. An inspection apparatus using magnetic resonance of claim 1 wherein the area other than the bone marrow area is the area where the reference sample is placed.

5. An inspection apparatus using magnetic resonance of the claim 4 wherein the reference sample is one of solution of sodium chloride, cupric sulfate, manganese chloride, gel of polyvinyl alcohol, vitamin D, or vitamin E.

6. An inspection apparatus using magnetic resonance of the claim 1 comprising an area specification means which specifies the bone marrow area of the flat bone or the long bone epiphysis, and/or the area other than the bone marrow area.

7. An inspection apparatus using magnetic resonance of the claim 1 comprising a decision device which determines the risk of bone density reduction of the object based on the result of the calculation.

8. An inspection apparatus using magnetic resonance of claim 7 wherein the decision device makes the decision by determining if the ratio of 1) the intensity of the nuclear magnetic resonance signal in the bone marrow area of the flat bone or the long bone epiphysis and 2) the intensity of the nuclear magnetic resonance signal in the area other than the bone marrow area is smaller or larger than a preliminarily given threshold.

9. An inspection apparatus using magnetic resonance of claim 7 wherein the decision device makes the decision by determining if the difference between 1) the intensity of the nuclear magnetic resonance signal in the bone marrow area of the flat bone or the long bone epiphysis and 2) the intensity of the nuclear magnetic resonance signal in the area other than the bone marrow area is smaller or larger than a preliminarily given threshold.

10. An inspection apparatus using magnetic resonance of claim 1 wherein the flat bone or the long bone epiphysis is one of a cranial bone, a sternum, a costa, a scapula, an ilium, a humerus epiphysis, or a femur epiphysis.

11. An inspection apparatus using magnetic resonance comprising:
a static magnetic field generating device which generates static magnetic field in an area in which an object is placed;
a gradient magnetic field generating device which gives gradient to the static magnetic field generated by the static magnetic field generating device;
a transmitter which applies RF pulse to the object;
a receiver which receives nuclear magnetic resonance signals generated from the object;
a control device which controls operation of the static magnetic field generating device, the gradient magnetic field generating device, the transmitter, and the receiver;
an area extraction means to automatically extract the bone marrow area of the flat bone or the long bone epiphysis from the nuclear magnetic resonance signal received by the receiver; and
an processing device which compares the intensity of the nuclear magnetic resonance signal in the bone marrow area of the flat bone or the long bone epiphysis received by the receiver, and the mean value of intensity of the nuclear magnetic resonance signal in the bone marrow area of the flat bone or the long bone epiphysis of a healthy control.

12. An inspection apparatus using magnetic resonance of claim 11, wherein the area extraction means extracts the bone marrow area by performing:
a first process to obtain a first image which comprises the brain area, the cerebrospinal fluid area, the scalp area, and the bone marrow area, by applying a high-pass filter with use of a first threshold to the nuclear magnetic resonance signal from the cranial bones of the object received by the receiver;
a second process to obtain a second image which comprises the brain area and the cerebrospinal fluid area, by applying a low-pass filter with use of a second threshold to the first image and extracting the maximum continuing area therefrom;
a third process to obtain a third image which comprises the scalp areas, by masking the first image by the second image and conducting the region growing method on the resultant image; and
a forth process to obtain a forth image which comprises the bone marrow area, by subtracting the second and third images from the first image to extract the bone marrow area.

13. An inspection apparatus using magnetic resonance of the claim 11, comprising an area specification means which specifies the bone marrow area of the flat bone or the long bone epiphysis, and/or the area other than the bone marrow area.

14. An inspection apparatus using magnetic resonance of the claim 11, comprising a decision device to determine the risk of bone density reduction of the object based on the result of calculation conducted by the processing device.

15. An inspection apparatus using magnetic resonance of claim 1, wherein the flat bone or the long bone epiphysis is one of a cranial bone, a sternum, a costa, a scapula, an ilium, a humerus epiphysis, or a femur epiphysis.

16. An inspection apparatus using magnetic resonance comprising:
a static magnetic field generating device which generates static magnetic field in an area in which an object is placed;
a gradient magnetic field generating device which gives gradient to the static magnetic field generated by the static magnetic field generating device;
a transmitter which applies RF pulse to the object;
a receiver which receives nuclear magnetic resonance signals generated from the object;
a control device which controls operation of the static magnetic field generating device, the gradient magnetic field generating device, the transmitter, and the receiver;
an area extraction means which automatically extracts the bone marrow area of the flat bone or the long bone epiphysis from the nuclear magnetic resonance signal received by the receiver;
a storage means which stores the nuclear magnetic resonance signal received by the receiver; and
an processing device which compares the intensity of the nuclear magnetic resonance signal in the bone marrow area of the flat bone or the long bone epiphysis at a first measurement and the intensity of the nuclear magnetic resonance signal in the bone marrow area of the flat bone or the long bone epiphysis at a second measurement.

17. An inspection apparatus using magnetic resonance of claim 16, wherein the area extraction means extracts the bone marrow area by performing:
a first process to obtain a first image which comprises the brain area, the cerebrospinal fluid area, the scalp area, and the bone marrow area, by applying a high-pass filter with use of a first threshold to the nuclear magnetic resonance signal from the cranial bones of the object received by the receiver;
a second process to obtain a second image which comprises the brain area and the cerebrospinal fluid area, by applying a low pass filter with use of a second threshold to the first image and extracting the maximum continuing area therefrom,
a third process to obtain a third image which comprises the scalp areas, by masking the first image by the second image and conducting the region growing method on the resultant image; and
a forth process to obtain a forth image which comprises the bone marrow area, by subtracting the second and third images from the first image to extract the bone marrow areas.

18. An inspection apparatus using magnetic resonance of the claim 16, comprising an area specification means which specifies the bone marrow area in the flat bone or the long bone epiphysis, and/or areas other than the bone marrow area.

19. An inspection apparatus using magnetic resonance of the claim 16, comprising a decision device to determine the risk of bone density reduction of the object based on the result of calculation conducted by the processing device.

20. An inspection apparatus using magnetic resonance of claim 16, wherein the flat bone or the long bone epiphysis is one of a cranial bone, a sternum, a costa, a scapula, an ilium, a humerus epiphysis, and a femur epiphysis.
